(19) United States
(12) Patent Application Publication
(10) Pub. No.: US 2013/0177591 A1
(43) Pub. Date: Jul. 11, 2013

(54) Lipid Based Adjuvants for DNA Plasmids

(71) Applicants: Alexis Parisot, Lyon (FR); Caroline Edlund Toulemonde, Villieu-Loyes-Moll (FR)

(72) Inventors: Alexis Parisot, Lyon (FR); Caroline Edlund Toulemonde, Villieu-Loyes-Moll (FR)

(73) Assignee: MERIAL LIMITED, Duluth, GA (US)

(21) Appl. No.: 13/738,550

(22) Filed: Jan. 10, 2013

Related U.S. Application Data
(60) Provisional application No. 61/584,874, filed on Jan. 10, 2012.

(51) Int. Cl. A61K 39/39; A61K 39/205

(52) U.S. Cl. CPC A61K 39/39; A61K 39/205
USPC 424/224.1

(57) ABSTRACT

The present invention provides for a novel vaccine formulation comprising plasmid DNA and a lipid adjuvant. The invention also provides for prime-boost vaccination methods wherein two sequential administration of the same plasmid-based DNA vaccine provide companion animals with protection against rabies for at least one year.

### CROSS-REFERENCE TO RELATED APPLICATIONS

This application claims benefit of U.S. Provisional Application No. 61/584,874, filed on Jan. 10, 2012, and incorporated herein by reference.

### FIELD OF THE INVENTION

The present invention relates to lipid-based compounds, their use as adjuvants for DNA vaccines, and pharmaceutical, immunologic, or vaccine compositions comprising the same.

### BACKGROUND

The use of adjuvants in vaccines is well known. An adjuvant is a compound that, when combined with a vaccine antigen, increases the immune response to the vaccine antigen as compared to the response induced by the vaccine antigen alone. Among strategies that promote antigen immunogenicity are those that render vaccine antigens particulate, those that polymerize or emulsify vaccine antigens, methods of encapsulating vaccine antigens, ways of increasing host innate cytokine responses, and methods that target vaccine antigens to antigen presenting cells (Nossal, 1999, In: Fundamental Immunology. Paul (Ed.), Lippincott-Raven Publishers, Philadelphia, Pa.; Vogel and Powell, 1995, In: Vaccine Design. The Subunit and Adjuvant Approach. Powell and Newman (Eds.), Plenum Press, NY, N.Y. p. 141). Adjuvants may, for example, consist of water-insoluble inorganic salts, liposomes, micelles or emulsions. Although there is no single mechanism of adjuvant action, an essential characteristic is their ability to significantly increase the immune response to a vaccine antigen as compared to the response induced by the vaccine antigen alone (Nossal, 1999, supra; Vogel and Powell, 1995, supra). In this regard, some adjuvants are more effective at augmenting humoral immune responses; other adjuvants are more effective at increasing cell-mediated immune responses (Vogel and Powell, 1995, supra); and yet another group of adjuvants increase both humoral and cell-mediated immune responses against vaccine antigens (Vogel and Powell, 1995, supra).

The use of deoxyribonucleic acid (DNA) molecules for vaccination has been known since the beginning of the 1990s (Wolf et al. Science 1990. 247. 1465-1468). This vaccination technique induces cellular and humoral immunity after in vivo transfection of cells of the subject to be vaccinated with DNA or RNA molecules encoding immunologically active proteins.

A DNA vaccine or immunogenic or immunological composition is composed of at least one plasmid which may be expressed by the cellular machinery of the subject to be vaccinated or inoculated and of a pharmaceutically acceptable vehicle or excipient. The nucleotide sequence of this plasmid encodes, inter alia, one or more immunogens, such as proteins or glycoproteins capable of inducing, in the subject to be vaccinated or inoculated, a cellular immune response (mobilization of the T lymphocytes) and a humoral immune response (stimulation of the production of antibodies specifically directed against the immunogen) (Davis H. L. Current Opinion Biotech. 1997. 8. 635-640).

All the immunogens derived from a pathogen are not antigens which are naturally sufficiently effective for inducing an optimum or protective immune response in the animal to be vaccinated or inoculated. It is therefore necessary to improve the immune response.

Various routes of administration of the DNA vaccine have been proposed (intraperitoneal, intravenous, intramuscular, subcutaneous, intradermal, mucosal, and the like). Various means of administration have also been proposed, in particular gold particles coated with DNA and projected so as to penetrate into the cells of the skin of the subject to be vaccinated (Tang et al. Nature 1992. 356. 152-154) and the liquid jet injectors which make it possible to transfect both skin cells and cells of the underlying tissues (Furth et al. Analytical Bioch. 1992. 205. 365-368).

Chemical compounds have been used for the in vitro transfection of DNA: A/—cationic lipids. The cationic lipids are themselves divided into four subgroups. 1) The cationic lipids containing quaternary ammonium salts, such as for example DOTMA (dioleoyl-oxypropyltrimethylammonium, produced by Gibco under the name Lipofectine), DOTAP (trimethyl-2,3-(octadec-9-eneoyloxy)-1-propaneammonium; Gregoriadis et al. FEBS Letters 1997. 402. 107-110), DMRIE (N-(2-hydroxyethyl)-N,N-dimethyl-2,3-bis(tetradecyloxy)-1-propaneammonium; WO-A-9634109), DLRIE (N-(2-hydroxyethyl)-N,N-dimethyl-2,3-bis(dodecyloxy)-1-propaneammonium; Felgner et al. Ann. N Y Acad. Sci. 1995. 772. 126-139).

These cationic lipids containing quaternary ammonium salts may be combined or otherwise with an additional neutral lipid, such as DOPC (dioleoylphosphatidylcholine) or DOPE (dioleoylphosphatidylethanolamine) (J. P. Behr, Bioconjugate Chemistry 1994. 5. 382-389). 2) The lipoamines, such as for example DOGS (dioctadecylamidoglycylspermine, produced by Promega under the name Transfectam; Abdallah et al. Biol. Cell. 1995. 85. 1-7), DC-Chol (dimethylaminoethane-carbamoyl-cholesterol; Gao and Huang, Biochem. Biophys. Res. Commun. 1991. 179. 280-285), BGSC (bis-guanidine-spermidine-cholesterol), BGTC (bis-guanidine-tren-cholesterol) (Vigneron et al. Proc. Natl. Acad. Sci. USA 1996. 93. 9682-9686). 3) The cationic lipids containing quaternary ammonium salts and lipoamines, such as for example DOSPA (N,N-dimethyl-N-(2-(sperminecarboxamido)ethyl)-2,3-bis(dioleoyloxy)-1-propaneimidium pentahydrochloride, marketed by Gibco under the name LipofectAmine®.; Hawley-Nelson et al. Focus 1993. 15. 73-79), GAP-DLRIE (N-(3-aminopropyl)-N,N-dimethyl-2,3-bis(dodecyloxy)-1-propaneammonium; Wheeler et al. Proc. Natl. Acad. Sci. USA 1996. 93. 11454-11459; Norman et al. Vaccine 1997. 15. 801-803). 4) The lipids containing amidine salts, such as for example ADPDE, ADODE (Ruysschaert et al. Biochem. Biophys. Res. Commun. 1994. 203. 1622-1628). B/—the polymers, such as for example SuperFect™ (molecules of activated dendrimers, produced by Qiagen; Xu et al. Mol. Genet. Metab. 1998. 64. 193-197), and C/—the biochemical agents, such as for example toxins, in particular cholera toxins.

Some of these compounds have also been used in the formulation of DNA vaccines with more than mitigated results. Knowledge in the field of in vitro transfection is not transposable to DNA vaccination where the final objective is to ensure an optimal and advantageously protective immune reaction. Negative effects on the induction of an effective immune protection have even been observed with compounds known to promote transfection in vitro. Some formulation chemical compounds are toxic at high doses for the transfected cells.

In the work by Etchart (Etchart et al. J. Gen. Virol. 1997. 78. 1577-1580), the use of DOTAP did not have an adjuvant effect during the administration of the DNA vaccine by the intranasal route, whereas it had an adjuvant effect by the oral route. DOTAP has also been used in DNA vaccines encoding the influenza virus hemagglutinin (HA) on the mouse model which were administered by the intranasal route (Ban et al. Vaccine 1997. 15. 811-813), but the addition of DOTAP inhibited the immune response. The use of DC-Chol or of DOTAP/DOPE in DNA vaccines encoding the hepatitis B virus surface protein (S) on the mouse model which were administered by the intramuscular route made it possible to increase the antibody response, whereas the use of Lipofectine (or DOTMA) did not increase this response (Gregoriadis et al. FEBS Letters 1997. 402. 107-110). DC-Chol/DOPE has also been used in DNA vaccines against the human immunodeficiency virus (HIV, Env protein) on the mouse model, whose administration by the intramuscular route induced a more effective immune response, whereas the administration by the subcutaneous or intradermal route did not increase it (Ishii et al. AIDS Res. Hum. Retro. 1997. 13. 1421-1428). Among above-recited compounds, DMRIE/DOPE (alone or in combination) have demonstrated particular utility in adjuvanting DNA vaccines (see for example U.S. Pat. No. 6,852,705, U.S. Pat. No. 7,078,388, each to Merial, and herein incorporated by reference in their entirety). Other effective adjuvanted DNA vaccines are described in U.S. Pat. No. 7,163,926 and U.S. Pat. No. 7,294,338

Moreover, it would be desirable to enhance or improve vaccination or immunization methods, for instance, the vaccination or immunization of companion animals including canines and felines; and, it would be desirable to provide vaccination or immunization methods involving a prime-boost regimen, as well as vaccines or immunological or immunogenic compositions, such as DNA vaccines or immunogenic or immunological compositions, which can be used such methods. However, until the present invention, few adjuvants have been demonstrated to effectively boost the immune responses elicited by DNA plasmid-based vaccines. There is, therefore, a need for novel adjuvanted formulations to fill this unmet need.

The foregoing applications, and all documents cited therein or during their prosecution (“applicant cited documents”) and all documents cited or referenced in the applicant cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### SUMMARY OF THE INVENTION

In a first embodiment the present invention provides for a novel immunogenic formulation comprising nucleic acids and a lipid adjuvant. In some embodiments, the lipid adjuvant is VAXFECTIN, as marketed and sold by Vical in 2011.

Yet another embodiment of the present invention provides for a stable, safe and injectable lipid formulation to increase the immune response induced by a immunogen. In particular, the present invention provides a novel adjuvant which, when used in a vaccine composition containing nucleic acid, particularly plasmid DNA, increases the vaccinate's cellular immune response, humoral immune response or, preferably, both to the immunogen.

A further embodiment of the present invention provides for a method of making a vaccine composition using the adjuvant of the instant invention; the vaccine composition so obtained; and methods of using thereof.

In another embodiment, the present invention provides for a method of inducing an immune response in a vaccinate against a pathogen comprising administering the vaccine composition of the present invention to the vaccinate.

The prime-boost regimen, according to the invention, can be used in animals of any age, advantageously young animals (e.g., animals that have detectable maternal antibodies and/or are suckling or nursing or breast-feeding, such as a young canine or feline that has detectable maternal antibodies and/or is suckling or nursing or breast-feeding), pre-adult animals (animals that are older than being a young animal but have not yet reached maturity or adulthood or an age to mate or reproduce), adult animals (e.g., animals that are of an age to mate or reproduce or are beyond such a period in life), and it is advantageous to employ the prime-boost regimen in pregnant females or females prior to giving birth or insemination.

The prime-boost regimen is especially advantageous to practice in a young animal, e.g., a young canine or feline, as it allows vaccination or immunization at an early age, for instance, the first administration in the prime-boost regimen or the prime can be administered to a young animal can be at an age at which the young animal has maternal antibodies. Another advantage of this regimen is that it can provide a degree of safety for pregnant females, e.g., canines or felines, present in the same location or in close proximity to the young or to each other.

Thus, the invention provides a prime-boost immunization or vaccination method advantageously practiced in canines and felines against one or more pathogens of canines and felines, and the method may be practiced upon a young animal, such as a young pup, for instance, wherein the priming is done at a time that the young animal has maternal antibodies against the canine or feline pathogen, with the boost advantageously at a time when maternal antibodies may be waning or decreasing or normally not present, such as during a period of time post-weaning.

The canine or feline pathogen against which the prime-boost regimen can be employed includes: rabies, canine parainfluenza (CPI), herpesvirus, canine distemper (CDV), feline calicivirus (FCV), feline infectious peritonitis, feline leukemia, and feline immunodeficiency virus. Advantageously, the prime-boost regimen of the invention is practiced against rabies virus.

The invention further comprehends the compositions and kits including one or more DNA vaccines or immunogenic or immunological compositions which may be used in the prime-boost regimen of the invention, and which make it possible to obtain an improved or advantageously effective and/or protective immune protection in canines comprising at least one valency selected from the group consisting of rabies, CPI, CAV, CCV, CDV, CIV, and parvovirus (comprising at least one plasmid that contains and expresses a nucleic acid molecule encoding at least one immunogen, antigen or epitope of rabies, CPI, CDV, CIV, and parvovirus). The canine pathogen may also be herpesvirus (CHV), canine parvovirus (CPV), canine coronavirus (CCV), Leptospira canicola, Leptospira icterohaemorragiae, Leptospira grippotyphosa, Leptospira pomona, Borrelia burgdorferi, Bordetella bronchiseptica.

The boosting vaccine or immunogenic or immunological composition is advantageously the same as the DNA vaccine or immunogenic or immunological composition.

Thus, the invention comprehends administering to a companion animal a priming composition comprising a DNA vaccine or immunogenic or immunological composition against a companion animal pathogen comprising at least one plasmid that contains and expresses in a companion animal host cell a nucleotide sequence encoding an immunogen, antigen or epitope of the companion animal pathogen, and thereafter a boosting composition that comprises the companion animal pathogen, or immunogen, antigen or epitope expressed by the DNA vaccine or immunogenic or immunological composition, that contains and expresses in a companion animal host cell a nucleotide sequence encoding the immunogen, antigen or epitope of the companion animal pathogen expressed by the DNA vaccine or immunogenic or immunological composition. The canine pathogen can be rabies glycoprotein G.

And, in general, DNA plasmids for DNA vaccines or immunogenic or immunological compositions, and DNA vaccines or immunogenic or immunological compositions employed in the “priming” and “boosting” of the herein prime-boost method may be as in U.S. Pat. No. 6,376,473 and U.S. application Ser. Nos. 10/085,519, 09/766,442, 09/760,574, 60/193,126, 09/232,468, 09/232,469, and 09/232,279, and French application No. 00 00798, filed Jan. 21, 2000.

These and other embodiments are disclosed or are obvious from and encompassed by, the following Detailed Description.

### BRIEF DESCRIPTION OF DRAWINGS

A full and enabling disclosure of the present invention, including the best mode thereof, to one of ordinary skill in the art, is set forth more particularly in the remainder of the specification, including reference to the accompanying figures, wherein:

FIG. 1 is a graph of rabies seroneutralization (SN, in IU/mL) as a function of days post-vaccination;

FIG. 2 is a graph of rabies SN at 42 days post infection, which indicates SN for group B is significantly higher than for groups A, D, and E.

### DETAILED DESCRIPTION OF THE INVENTION

Other objects, features and aspects of the present invention are disclosed in, or are obvious from, the following Detailed Description. It is to be understood by one of ordinary skill in the art that the present discussion is a description of exemplary embodiments only and is not intended as limiting the broader aspects of the present invention, which broader aspects are embodied in the exemplary construction. In fact, it will be apparent to those skilled in the art that various modifications and variations can be made in the present invention without departing from the scope or spirit of the invention. For instance, features illustrated or described as part of one embodiment can be used in another embodiment to yield a still further embodiment. It is intended that the present invention cover such modifications and variations as come within the scope of the appended claims and their equivalents. The contents of all references, published patents, and patents cited throughout the present application are hereby incorporated by reference in their entirety.

For convenience, certain terms employed in the Specification, Examples, and appended Claims are collected here.

It is noted that in this disclosure and particularly in the claims, terms such as “comprises”, “comprised”, “comprising” and the like can have the meaning attributed to such terms in U.S. patent law; e.g., they can mean “includes”, “included”, “including”, and the like; and that terms such as “consisting essentially of” and “consists essentially of” have the meaning ascribed to them by U.S. patent law, e.g., they allow for elements not explicitly recited, but exclude elements that are found in the prior art or that affect a basic or novel characteristic of the invention.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

As used herein, the term “animal” includes all vertebrate animals including humans. It also includes an individual animal in all stages of development, including embryonic and fetal stages. In particular, the term “vertebrate animal” includes, but not limited to, humans, canines (e.g., dogs), felines (e.g., cats); equines (e.g., horses), bovines (e.g., cow, cattle), porcine (e.g., pigs), as well as in avians. As used herein, the term “dog” or “canine” is used generally to refer to an animal of canine origin of any age. Likewise, the term “cat” or “feline” is used generally to refer to an animal of feline origin of any age. The term “avian” as used herein refers to any species or subspecies of the taxonomic class ava, such as, but not limited to, chickens (breeders, broilers and layers), turkeys, ducks, a goose, a quail, pheasants, parrots, finches, hawks, crows and ratites including ostrich, emu and cassowary. The term “pig” or “piglet” means an animal of porcine origin, while “sow” refers to a female of reproductive age and capability.

The terms “immunogenic composition” and “immunological composition” and “immunogenic or immunological composition” cover any composition that elicits an immune response against the targeted pathogen; for instance, after administration or injection into the canine elicits an immune response against the targeted pathogen. The terms “vaccinal composition” and “vaccine” and “vaccine composition” covers any composition that induces a protective immune response against the targeted pathogen or which efficaciously protects against the pathogen; for instance, after administration or injection into the canine, elicits a protective immune response against the targeted pathogen or provides efficacious protection against the pathogen. Furthermore, while the text speaks of “immunogen, antigen or epitope”, an immunogen can be an antigen or an epitope of an antigen.

The term of “prime-boost” refers to the successive administration of vaccine or immunogenic or immunological compositions having at least one immunogen, antigen or epitope in common. The priming administration (priming) is the administration of a first vaccine or immunogenic or immunological composition type and may comprise one, two or more administrations. The boost administration is the administration of a second vaccine or immunogenic or immunological composition type and may comprise one, two or more administrations, and, for instance, may comprise or consist essentially of annual administrations.

As used herein, the term “virulent” means an isolate that retains its ability to be infectious in an animal host.

As used herein, the term “inactivated vaccine” means a vaccine composition containing an infectious organism or pathogen that is no longer capable of replication or growth. The pathogen may be bacterial, viral, protozoal or fungal in origin. Inactivation may be accomplished by a variety of methods including freeze-thawing, chemical treatment (for example, treatment with formalin), sonication, radiation, heat or any other convention means sufficient to prevent replication or growth of the organism while maintaining its immunogenicity.

As used herein, the term “immunogenicity” means capable of producing an immune response in a host animal against an antigen or antigens. This immune response forms the basis of the protective immunity elicited by a vaccine against a specific infectious organism.

As used herein, the term “immune response” refers to a response elicited in an animal. An immune response may refer to cellular immunity (CMI); humoral immunity or may involve both. The present invention also contemplates a response limited to a part of the immune system. For example, a vaccine composition of the present invention may specifically induce an increased gamma interferon response.

As used herein, the term “antigen” or “immunogen” means a substance that induces a specific immune response in a host animal. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a protein, a polypeptide, a peptide, an epitope, a hapten, or any combination thereof. Alternately, the immunogen or antigen may comprise a toxin or antitoxin.

As used herein, the term “multivalent” means a vaccine containing more than one antigen whether from the same species (i.e., different isolates of rabies or CDV virus serotypes), from a different species (i.e., isolates from both leptospira canicola and leptospira icterohaemorrhagiae), or a vaccine containing a combination of antigens from different genera.

As used herein, the term “adjuvant” means a substance added to a vaccine to increase a vaccine's immunogenicity. Known vaccine adjuvants include, but are not limited to, oil and water emulsions, Corynebacterium parvum, Bacillus Calmette Guerin, aluminum hydroxide, glucan, dextran sulfate, iron oxide, sodium alginate, Bacto-Adjuvant, certain synthetic polymers such as poly amino acids and co-polymers of amino acids, saponin, “REGRESSIN” (Vetrepharm, Athens, Ga.), “AVRIDINE” (N,N-dioctadecyl-N′,N′-bis(2-hydroxyethyl)-propanediamine), VAXFECTIN (Vical), paraffin oil, muramyl dipeptide and the like.

As used herein, the term “emulsion” refers to a combination of at least two substances, wherein a first substance is dispersed in a second substance in which the first substance is insoluble. One example of an emulsion of the present invention is an oil phase dispersed in an aqueous phase.

As used herein, the terms “pharmaceutically acceptable carrier” and “pharmaceutically acceptable vehicle” are interchangeable and refer to a fluid vehicle for containing vaccine antigens that can be injected into a host without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

As used herein, the term “vaccine composition” includes at least one antigen or immunogen in a pharmaceutically acceptable vehicle useful for inducing an immune response in a host. Vaccine compositions can be administered in dosages and by techniques well known to those skilled in the medical or veterinary arts, taking into consideration such factors as the age, sex, weight, species and condition of the recipient animal, and the route of administration. The route of administration can be percutaneous, via mucosal administration (e.g., oral, nasal, anal, vaginal) or via a parenteral route (intradermal, intramuscular, subcutaneous, intravenous, or intraperitoneal). Vaccine compositions can be administered alone, or can be co-administered or sequentially administered with other treatments or therapies. Forms of administration may include suspensions, syrups or elixirs, and preparations for parenteral, subcutaneous, intradermal, intramuscular or intravenous administration (e.g., injectable administration) such as sterile suspensions or emulsions. Vaccine compositions may be administered as a spray or mixed in food and/or water or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired. Standard pharmaceutical texts, such as “Remington's Pharmaceutical Sciences,” 1990 may be consulted to prepare suitable preparations, without undue experimentation.

The term “purified” as used herein does not require absolute purity; rather, it is intended as a relative term. Thus, for example, a purified immunogen preparation, such as protein or inactivated virus, is one in which the immunogen is more enriched than the immunogen is in its natural environment. An immunogen preparation is herein broadly referred to as “purified” such that the immunogen represents at least 60%, at least 70%, at least 80%, at least 90%, at least 95%, or at least 98%, of the total immunogen content of the preparation. A “crude preparation”, which represents the lowest degree of purification, may contain as little as less than 60%, lest than 20%, less than 10%, less than 5%, or less than 1% of immunogenic components.

The term “highly purified” as used herein is intended to suggest a “higher degree of purity” as compared to the term “moderately purified”. This “higher degree of purity” can include, but is in no way limited to, reduced percentages of contaminants, in an immunological preparation that has been “highly purified” versus an immunological preparation that has been “moderately purified”. As discussed herein, “highly purified” immunological preparations will have the lowest to undetectable percentages of contaminants that can cause: reduced desired immune response, increased undesired immune response (e.g. hypersensitivity reaction), or reduced formulation stability. Similarly, an immunological preparation that has been “moderately purified” contains relatively reduced percentages of contaminants versus an immunological preparation that has been “minimally purified”, which likewise, has reduced percentages of contaminants versus a preparation designated a “crude preparation”.

Contaminants in an immunological preparation can include, but are in no way limited to, substances that contribute negatively to an immunological composition according to the present invention. One of several examples of a contaminant contributing negatively would be a contaminant that reduces the ability of an immunological composition of the present invention to elicit an immune response in animals.

The immunogen or antigen suitable for use in the present invention may be selected from the group consisting of nucleotides (e.g. plasmid DNA), inactivated pathogens, attenuated pathogens, immunogenic sub-units (e.g. proteins, polypeptides, peptides, epitopes, haptens), and recombinant expression vectors. In one embodiment of the present invention, the immunogen is an inactivated or killed microorganism. In one embodiment, a vaccine composition of the present invention comprises an immunogen selected from a canine pathogen including, but not limited to, rabies virus, CPI, CAV, CCV, CDV, CIV, canine herpesvirus (CHV), canine parvovirus (CPV), canine coronavirus (CCV), Leptospira canicola, Leptospira icterohaemorragiae, Leptospira grippotyphosa, Leptospira pomona, Borrelia burgdorferi, Bordetella bronchiseptica and the like, and combinations thereof. Alternately, the vaccine composition comprises an immunogen selected from a feline pathogen such as, but not limited to, feline herpesvirus (FHV), feline calicivirus (FCV), feline leukemia virus (FeLV), feline immunodeficiency virus (FIV), rabies virus, and the like, and combinations thereof.

In another embodiment of the invention, the vaccine composition comprises an immunogen selected from the group of avian pathogens including, but not limited to, Salmonella typhimurium, Salmonella enteritidis, Infectious Bronchitis virus (IBV), Newcastle Disease virus (NDV), egg drop syndrome virus (EDS), or Infectious Bursal Disease virus (IBDV), avian influenza virus, and the like, and combinations thereof.

In yet another embodiment of the invention the composition comprises an immunogen selected from an equine pathogen, such as equine herpesvirus (type 1 or type 4), equine influenza virus, tetanus, west nile virus, and the like or combinations thereof. In another embodiment of the invention, the composition comprises an immunogen selected from an bovine pathogen, such as foot and mouth disease virus (FMDV), rabies virus, bovine rotavirus, bovine parainfluenza virus type 3 (bPIV-3), bovine coronavirus, bovine viral diarrhea virus (BVDV), bovine respiratory syncytial virus (BRSV), Infectious Bovine Rhinotracheitis virus (IBR), Escherichia coli, Pasteurella multocida, Pasteurella haemolytica and the like and combinations thereof.

In still another embodiment of the present invention, the composition comprises an immunogen selected from an porcine pathogen such as, but not limited to, swine influenza virus (SIV), porcine circovirus type 2 (PCV-2), porcine reproductive respiratory syndrome virus (PRRS), pseudorabies virus (PRV), porcine parvovirus (PPV), FMDV, Mycoplasma hyopneumoniae, Erysipelothrix rhusiopathiae, Pasteurella multocida, Bordetella bronchiseptica, Escherichia coli and the like, and combinations thereof.

Another embodiment of the invention provides for vaccine compositions comprising at least one immunogen and a lipid adjuvant in a pharmaceutically acceptable vehicle. Immunogens useful in vaccine compositions according to the present invention include expression vectors. Such vectors include, but are not limited to, in vivo recombinant expression vectors such as a polynucleotide vector or a plasmid (EP-A2-1001025; Chaudhuri P, Res. Vet. Sci. 2001, 70: 255-6), virus vectors such as, but not limited to, adenovirus vectors, poxvirus vectors such as fowlpox (U.S. Pat. Nos. 5,174,993; 5,505,941; and 5,766,599) or canarypox vectors (U.S. Pat. No. 5,756,103) or bacterial vectors (Escherichia coli or Salmonella sp.)/

The present invention further provides for methods for inducing an immune response in a host, e.g., an animal, comprising administering to the host an immunological composition or a vaccine composition according to the invention. The immune responses elicited in this manner are notably antibody and/or cellular immune responses, and in particular, a gamma-interferon response.

In particular, the present invention provides for methods to immunize against, or to prevent or to reduce the symptoms caused by, infection of an animal with a pathogenic organism (for example, infection by a virus, bacteria, fungus, or protozoan parasite). The method of the present invention is useful in vertebrate animals including, but not limited to, humans, canines (e.g., dogs), felines (e.g., cats); equines (e.g., horses), bovines (e.g., cattle) and porcine animals (e.g., pigs), as well as in avians including, but not limited to, chickens, turkeys, ducks, geese, a quail, a pheasant, parrots, finches, hawks, crows and ratites (ostrich, emu, cassowary, and the like).

In a particular aspect of the invention, these methods consist of the vaccination of pregnant females before parturition by administering a vaccine composition made according to the invention. These methods further include the induction of protective antibodies elicited by the vaccination protocol and the transfer of these protective antibodies from vaccinated pregnant females to their offspring. The transfer of such maternal antibodies subsequently protects the offspring from disease.

The dosage of the vaccine composition made according to the present invention will depend on the species, breed, age, size, vaccination history, and health status of the animal to be vaccinated. Other factors like antigen concentration, additional vaccine components, and route of administration (i.e., subcutaneous, intradermal, oral, intramuscular or intravenous administration) will also impact the effective dosage. The dosage of vaccine to administer is easily determinable based on the antigen concentration of the vaccine, the route of administration, and the age and condition of the animal to be vaccinated. Each batch of antigen may be individually calibrated. Alternatively, methodical immunogenicity trials of different dosages, as well as LD₅₀ studies and other screening procedures can be used to determine effective dosage for a vaccine composition in accordance with the present invention without undue experimentation. From the examples presented below, it will be readily apparent what approximate dosage and what approximate volume would be appropriate for using the vaccine composition described herein. The critical factor is that the dosage provides at least a partial protective effect against natural infection, as evidenced by a reduction in the mortality and morbidity associated with natural infection. The appropriate volume is likewise easily ascertained by one of ordinary skill in the art. For example, in avian species the volume of a dose may be from about 0.1 ml to about 0.5 ml and, advantageously, from about 0.3 ml to about 0.5 ml. For feline, canine and equine species, the volume of a dose may be from about 0.2 ml to about 3.0 ml, advantageously from about 0.3 ml to about 2.0 ml, and more advantageously, from about 0.5 ml to about 1.0 ml. For bovine and porcine species, the volume of dose may be from about 0.2 ml to about 5.0 ml, advantageously from about 0.3 ml to about 3.0 ml, and more advantageously from 0.5 ml to about 2.0 ml.

Repeated vaccinations may be preferable at periodic time intervals to enhance the immune response initially or when a long period of time has elapsed since the last dose. In one embodiment of the present invention, the vaccine composition is administered as a parenteral injection (i.e., subcutaneously, intradermally, or intramuscularly). The composition may be administered as one dose or, in alternate embodiments, administered in repeated doses of from about two to about five doses given at intervals of about two to about six weeks, preferably from about two to about five weeks. However, one of skill in the art will recognize that the number of doses and the time interval between vaccinations depends on a number of factors including, but not limited to, the age of the animal vaccinated; the condition of the animal; the route of immunization; amount of antigen available per dose; and the like. For initial vaccination, the period will generally be longer than a week and preferably will be between about two to about five weeks. For previously vaccinated animals, a booster vaccination, before or during pregnancy, at about an annual interval may be performed.

The present invention also contemplates administering a vaccine composition using a needle free injector such as PIGJET®, AVIJET®, DERMOJET® or BIOJECTOR® (Bioject, Oregon, USA). An person of ordinary skill in the art is able to adjust the specifications of the injector as required with regard to factors such as the species of the animal to be vaccinated; the age and weight of the animal, and the like without undue experimentation.

In one embodiment of the present invention, the method comprises two sequential administrations of a DNA vaccine composition formulated with a lipid adjuvant according to the invention. For example, in one embodiment, the vaccine composition comprises pVR1012 plasmid harboring the rabies G nucleic acid sequence.

The invention will now be further described by way of the following non-limiting examples.

### Example 1

### Vaccination of Dogs

Two studies were conducted in dogs to evaluate lipid based adjuvants for DNA plasmids. Fifty micrograms of plasmid DNA was injected either by IM or SC route in prime-boost vaccination regime (Day 0 and Day 29). The model plasmid DNA used in these studies was pPB266. Additional details on Rabies G may be found, for example, in U.S. Pat. No. 7,163,926 and U.S. Pat. No. 7,294,338 (to Merial), which are herein incorporated by reference in their entirety.

Description of Adjuvants.

VAXFECTIN is a cytofectin:co-lipid mixture of GAP-DMORIE/DPyPE at a 1:1 molar ratio. Rather than employing the more cumbersome formal chemical nomenclature and stipulating the specific molar ratio for the mixture, this novel formulation has been named “Vaxfectin” (please see U.S. Pat. No. 7,582,613 to Vical Inc.). The adjuvant and diluent are maintained at a temperature below −10° C.

ADJUVANT N3 (Hinkula et al., Vaccine V.24, May 2006) is a cationic additive based on endogenous lipid (oleyl amine). The formulation generates nano-droplets of oil (single unilamellar vesicles, SUV). Its cationic charge promotes interaction with anionic antigens such as DNA. N3 may be prepared according to the publication: “To obtain a 4% N3 lipid emulsion, 0.4 g of the 1:1 (molar ratio) mixture of mono-olein and oleylamine, 9.6 ml 0.1 M Tris-buffer, pH 8.0 was added to a beaker. The N3 emulsion was formed by sonication for 2 min, the pH was adjusted to 8.0. The final vaccine formulation was a 1:1 (v/v) mixture of the obtained N3 emulsion and a DNA solution having a concentration of 12 μg DNA/ml. The different doses of the N3 adjuvant was obtained by dilutions of the prepared 4% N3 lipid emulsion.” The data showed that the adjuvant increased the immunogenicity of DNA vaccines by the nasal route. The adjuvant is stored at a temperature of about 5° C. (diluent at room temperature).

ICA 614 and ICA 614-M are amphiphilic block copolymers (please see U.S. Pat. No. 7,709,452 and US 2010/0179212 A1, to INSERM, which are herein incorporated by reference in their entirety). The hydrophobic core of the molecule is composed of propylene oxide and the hydrophilic part is composed of ethylene oxide. The molecule has a star conformation with four arms (tetrafunctional). The adjuvant and diluent are stored at a temperature below −20° C.

TABLE 1
Experimental Plan

| Groups | | | |
|---|---|---|---|
| (n = 6) | Antigen | Adjuvant | Route |
| A | DNA pPB266 (50 μg/ml) | N/A | IM or SC |
| B | DNA pPB266 (50 μg/ml) | Formulation I: VAXFECTIN ® | IM or SC |
| C | DNA pPB266 (50 μg/ml) | Formulation II: N3-adjuvant | IM or SC |
| D | DNA pPB266 (50 μg/ml) | Formulation III: ICA 614 | IM or SC |
| E | DNA pPB266 (50 μg/ml) | Formulation IV: ICA 614-M | IM or SC |
| F | DNA pVR1012 (50 μg/ml) − control | | |

I. Cationic liposome from Vical (Vaxfectin)
II. Endogenous cationic lipid based adjuvant (N3 = Oleyl amine) from Eurocine
III. Amphiphilic block co-polymer from InCellArt
IV. Amphiphilic block co-polymer + Mannose from InCellArt

TABLE 2
Study Design - IM route

| | Formulations | First Vaccination | Blood | | | |
|---|---|---|---|---|---|---|
| | tested | 1 ml by IM in | samples | Blood samples | | Monitoring of |
| Group | (50 μg/ml DNA) | tibialis cranialis | for serology | for CMI | Clinical Monitoring | weight |
| A (n = 6) | naked DNA | D0 (V1) & | D0*, D28, | D31, D37, | D0*, D0 +4 h, D1, D2, D3, | D-4 |
| | (pPB266) | D29 (V2) | D44 & | D44, D58 & D65 | D4 & D7 | |
| | | | D58 | | | |
| B (n = 6) | I | | | | D29*, D29 +4 h, | D28 |
| | | | | | D31, D32, D35 & D36 | |
| C (n = 6) | II | | | | | |
| D (n = 6) | III | | | | | D57 |
| E (n = 6) | IV | | | | | |
| F (n = 6) | control DNA | | D0 & | | | |
| | (pVR1012) | | D58 | | | |

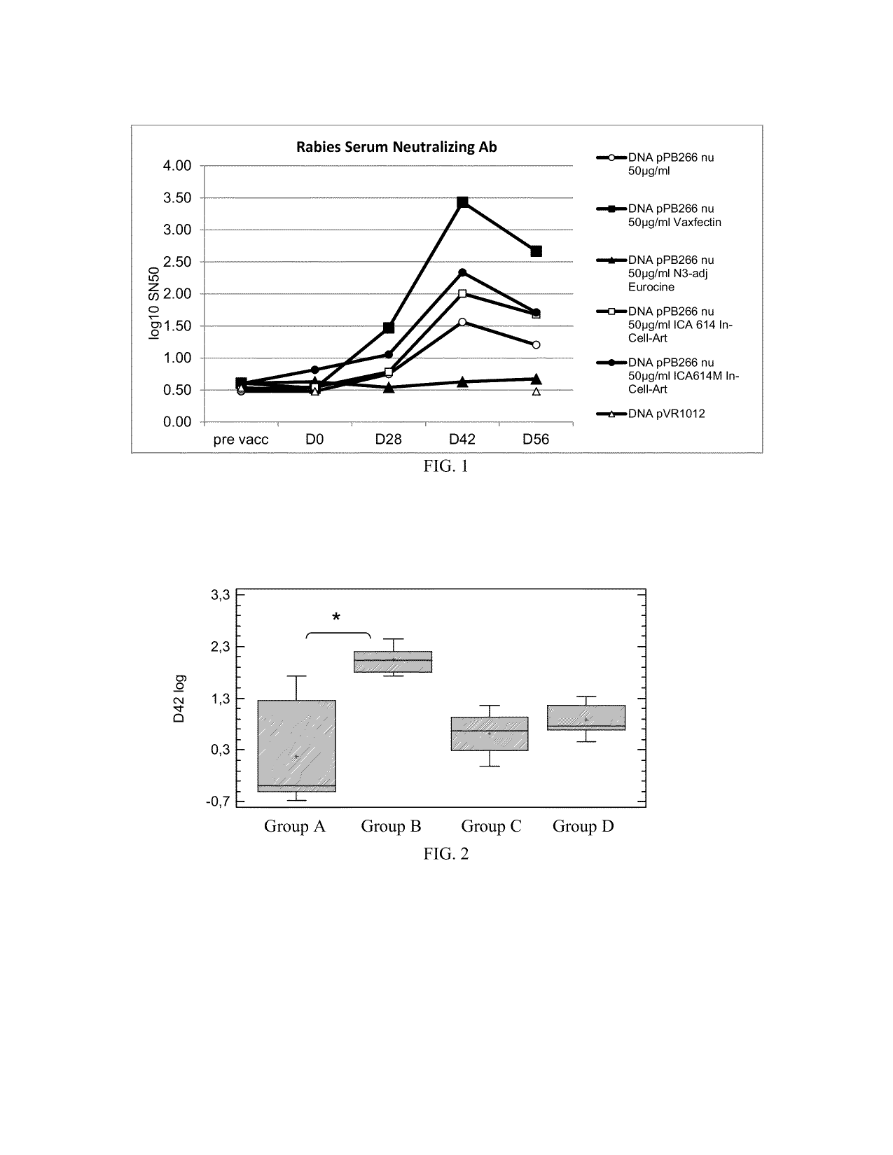

The study result showed no systemic adverse events in group B (VAXFECTIN®) after vaccinations. FIG. 1 shows the surprising results that a huge boost effect upon the second immunization, with anti-rabies SN titers reaching more than 150 IU/ml and above in group B (VAXFECTIN®). The serology result in FIG. 2 shows that the titer in group B is significantly higher than titers in groups A, D and E. Thus, in an embodiment, the present disclosure provides a prime-boost method for vaccinating an animal against rabies comprising administering to said animal, on two separate occasions, an adjuvanted vaccine composition comprising (a) nucleotides encoding a rabies G protein; and (b) a lipid adjuvant comprising GAP-DMORIE (1,2-diphytanoyl-sn-glycero-3-phosphoethanolamine) and DPyPE (1,2-diphytanoyl-sn-glycero-3-phosphoethanolamine). In a particular embodiment (as indicated by the data presented in FIG. 1), the animals vaccinated with the VAXFECTIN-adjuvanted rabies G DNA plasmid vaccine composition produce more than twice as many serum-neutralizing antibodies as animals vaccinated with an identical amount the rabies G DNA plasmid vaccine composition, but containing no adjuvant.

What is claimed is:

1. A prime-boost method for vaccinating an animal against rabies comprising administering to said animal, on two separate occasions, an adjuvanted vaccine composition comprising (a) nucleotides encoding a rabies G protein; and (b) a lipid adjuvant comprising GAP-DMORIE (1,2-diphytanoyl-sn-glycero-3-phosphoethanolamine) and DPyPE (1,2-diphytanoyl-sn-glycero-3-phosphoethanolamine).

2. The method of claim 1 wherein the two administrations provide protection against rabies for at least one year following the second administration of the vaccine.

3. The method of claim 1 wherein the nucleotides comprise from 10 ng to 1 mg of plasmid DNA.

4. The method of claim 3 wherein the nucleotides comprise from 100 ng to 500 μg of the plasmid.

5. The method of claim 4 wherein the nucleotides comprise from 1 μg and 250 μg of the plasmid.

6. The method of claim 1 or 5 wherein the vaccine is administered in a dose volume between 0.1 and 5 ml.

7. The method of claim 6 wherein the vaccine is administered in a dose volume between 0.5 and 2 ml.

8. The method of claim 1 or 5 wherein the vaccinated animals produce more than twice as many serum-neutralizing antibodies as animals vaccinated with an identical amount the nucleotides encoding the rabies G protein, but containing no adjuvant.

9. An immunological or vaccine composition comprising a plasmid, which contains and expresses in a non-human animal host a rabies G protein, and a lipid adjuvant comprising GAP-DMORIE (1,2-diphytanoyl-sn-glycero-3-phosphoethanolamine) and DPyPE (1,2-diphytanoyl-sn-glycero-3-phosphoethanolamine).

10. The composition of claim 9 wherein the lipid adjuvant is VAXFECTIN.

11. The composition of claim 9 wherein the lipid:DNA ratio is between 0.5-1.5 to 0.5-1.5.

12. The composition of claim 11 wherein the lipid:DNA ratio is between 0.75-1.25 to 0.75-1.25.

13. The composition of claim 12 wherein the lipid:DNA ratio is about 1:1.
